(19) United States
(12) Patent Application Publication
(10) Pub. No.: US 2013/0171200 A1
(43) Pub. Date: Jul. 4, 2013

(54) Heat Patch for Diabetes

(71) Applicant: Tuming You, Xiamen (CN)

(72) Inventor: Tuming You, Xiamen (CN)

(21) Appl. No.: 13/552,507

(22) Filed: Jul. 18, 2012

Related U.S. Application Data
(63) Continuation-in-part of application No. 13/471,138, filed on May 14, 2012.
Application No. 13/471,138 is a continuation-in-part of application No. 13/341,876, filed on Dec. 30, 2011.

(51) Int. Cl. A61K 9/00; A61K 33/24; A61K 35/55; A61K 31/045; A61K 36/28; A61P 3/10; A61K 36/63

(52) U.S. Cl. 424/400; 424/617; 424/769; 514/729; 424/725; 424/525

(57) ABSTRACT

Provided in the present invention are a composition for ameliorating or treating a diabetic condition and methods of making and using the same.

### CROSS-REFERENCE TO RELATED APPLICATIONS

This application is a continuation-in-part of U.S. application Ser. No. 13/471,138, filed on May 14, 2012. This application is also a continuation-in-part of U.S. application Ser. No. 13/341,876, filed on Dec. 30, 2011. The teachings in these applications are incorporated herein in their entirety by reference.

### FIELD OF THE INVENTION

The invention is related to methods and articles for ameliorating or treating a diabetic condition.

### BACKGROUND OF THE INVENTION

Diabetes is a major health concern of modern human life. Diabetes is a lifelong (chronic) disease in which there are high levels of sugar in the blood. Insulin is a hormone produced by the pancreas to control blood sugar. Diabetes can be caused by too little insulin, resistance to insulin, or both. People with diabetes have high blood sugar because their body cannot move sugar into fat, liver, and muscle cells to be stored for energy. This is because either: (1) their pancreas does not make enough insulin, (2) their cells do not respond to insulin normally, or (3) both.

There are three major types of diabetes. The causes and risk factors are different for each type:

(1) Type 1 diabetes can occur at any age, but it is most often diagnosed in children, teens, or young adults. In this disease, the body makes little or no insulin. Daily injections of insulin are needed. The exact cause is unknown.

(2) Type 2 diabetes makes up most of diabetes cases. It most often occurs in adulthood, but teens and young adults are now being diagnosed with it because of high obesity rates. Many people with type 2 diabetes do not know they have it.

(3) Gestational diabetes is high blood sugar that develops at any time during pregnancy in a woman who does not have diabetes.

Diabetes affects more than 20 million Americans. Over 40 million Americans have pre-diabetes (early type 2 diabetes).

According to traditional Chinese medicine, blocking of the free flow of human biological system happens in certain places (points) in the human body and cause diseases to occur. A particular disorder corresponds to blocking of a particular point or set of points in the biological system. Stimulating one of these points can free up the flow of biological system of human being so as to treat or ameliorate a disease arising out of blocking of such point or set of points. According to this school of medicine, a method of therapeutics —acupuncture—was developed and practiced in China for thousands of years. The oldest medical book known, written in China 4000 years ago, describes the use of acupuncture to treat medical problems. The use of the treatment spread to other Asian countries and to other regions of the world, including to Europe by the 1700s. In the United States, acupuncture has been used for about 200 years.

Acupuncture has long been successfully used in treatment of diabetes (see, e.g., Abuaisha, et al., Diabetes Research and Clinical Practice, 39(2), Pages 115-121, (1998). A common side effect of acupuncture is concern of infection caused by cross-contamination of acupuncture needles that may include viral or bacterial pathogens. Other concerns may arise out of patient's negative reactions against piercing of acupuncture needles or misapplication of such needles by a practitioner. Further, access to acupuncture by a practitioner is limited so as to limit the effect of acupuncture.

Therefore, there is a need for a different way of acupuncture treating or management paradigm for diabetes.

The embodiments described below address the above identified needs and issues.

### SUMMARY OF THE INVENTION

In one aspect of the present invention, it is provided a composition for diabetes, the composition comprises a heat generation component, the composition is formulated in a formulation for selective application to a set of acupoints or one or more acupoint(s) for diabetes (“acupoint(s) for diabetes”) in a patient, and the composition generates heat upon application to the acupoint(s) for diabetes for a sufficient period of time to generate an effect to ameliorate or treat diabetes.

In some embodiments of the composition, the set of acupoints comprises the Shangwan, Zhongwan, Shenque, Zusanli, Weizhong, Ganshu, Danshu, and Shenshu acupoints of a diabetes patient.

In some embodiments of the composition, optionally in combination with any or all of the above various embodiments, the composition is capable of generating heat for at least 1 hour (e.g., 1 hour, 2 hours, 4 hours, 8 hours, or 10 hours) after application to the set of acupoints or one or more acupoint(s) for diabetes.

In some embodiments of the composition, optionally in combination with any or all of the above various embodiments, the heat generation component comprises a rare earth component.

In some embodiments of the composition, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of the composition, optionally in combination with any or all of the above various embodiments, the formulation is a patch comprising a cover. In some embodiments, the cover is a plastic, rubber, or paper cover.

In another aspect of the present invention, it is provided a method of fabricating a composition for diabetes, comprising providing a heat generation component, and formulating the composition in a formulation for selective application to a set of acupoints or one or more acupoint(s) for diabetes (“acupoint(s) for diabetes”) in a patient. The composition generates heat upon application tope set of acupoints or one or more acupoint(s) for diabetes for a sufficient period of time to generate an effect to ameliorate or treat diabetes.

In some embodiments of the method, the set of acupoints comprises the Shangwan, Zhongwan, Shenque, Zusanli, Weizhong, Ganshu, Danshu, and Shenshu acupoints of a diabetes patient.

In some embodiments of method, optionally in combination with any or all of the above various embodiments, the composition is capable of generating heat for at least 1 hour (e.g., 1 hour, 2 hours, 4 hours, 8 hours, or 10 hours) after application to the set of acupoints or one or more acupoint(s) for diabetes.

In some embodiments of method, optionally in combination with any or all of the above various embodiments, the heat generation component comprises a rare earth component.

In some embodiments of method, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of method, optionally in combination with any or all of the above various embodiments, the formulation is a patch comprising a cover. In some embodiments, the cover is a plastic, rubber, or paper cover.

In a further embodiment of the present invention, it is provided a method of ameliorating or treating a diabetic condition, comprising applying a composition at least once per day to a set of acupoints or one or more acupoint(s) for diabetes (“acupoint(s) for diabetes”) in a diabetes patient, which composition comprising a heat generation component, wherein the composition is formulated in a formulation for selective application to the set of acupoints or one or more acupoint(s) for diabetes over the entire length of a prescribed treatment course, wherein the composition generates heat upon application to the set of acupoints or one or more acupoint(s) for diabetes for a sufficient period of time to generate an effect to ameliorate diabetes.

In some embodiments of the method, the set of acupoints comprises the Shangwan, Zhongwan, Shenque, Zusanli, Weizhong, Ganshu, Danshu, and Shenshu acupoints of a diabetes patient.

In some embodiments of method, optionally in combination with any or all of the above various embodiments, the composition is capable of generating heat for at least 1 hour (e.g., 1 hour, 2 hours, 4 hours, 8 hours, or 10 hours) after application to the set of acupoints or one or more acupoint(s) for diabetes.

In some embodiments of method, optionally in combination with any or all of the above various embodiments, the heat generation component comprises a rare earth component.

In some embodiments of method, optionally in combination with any or all of the above various embodiments, the heat generation component comprises a rare earth magnetic powder, saussurea in volucrata, musk, sea otter chuanwu, caowu, lilac, menthol, meiyao, wujiapi, and magianzhi.

In some embodiments of method, optionally in combination with any or all of the above various embodiments, the formulation is a patch comprising a cover. In some embodiments of method, the cover is a plastic, rubber, or paper cover.

In some embodiments of method, optionally in combination with any or all of the above various embodiments, the treatment course lasts for about 1 month, 3 months, 6 months, or 12 months.

### BRIEF DESCRIPTION OF THE DRAWINGS

FIGS. 1A-1D show acupoints for diabetes as embodiments of the present invention.

### DESCRIPTION OF EXEMPLARY EMBODIMENTS OF THE INVENTION

In one aspect of the present invention, it is provided a composition for diabetes, the composition comprises a heat generation component, the composition is formulated in a formulation for selective application to a set of acupoints or one or more acupoint(s) for diabetes (“acupoint(s) for diabetes”) in a patient, and the composition generates heat upon application to the acupoint(s) for diabetes for a sufficient period of time to generate an effect to ameliorate or treat diabetes. As used herein, the term diabetes includes the disorder as diagnosed by general medical standards and symptoms of diabetes.

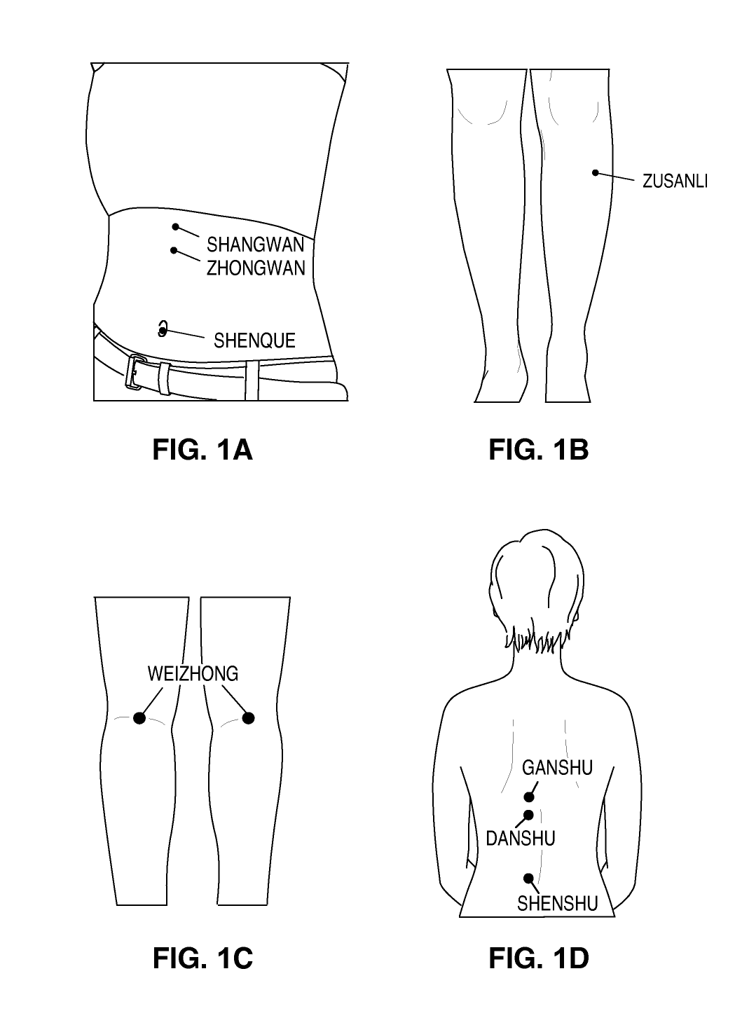

the set of acupoints comprises the Shangwan, Zhongwan, Shenque, Zusanli, Weizhong, Ganshu, Danshu, and Shenshu acupoints of a diabetes patient (FIGS. 1A-1D).

In some embodiments, optionally in combination with any or all of the above various embodiments, the composition is capable of generating heat for at least 10 minutes, e.g., 20 minutes, 30 minutes, 60 minutes (1 hour), 2 hours, 3 hours, 4 hours, 5 hours, 6 hours, 7 hours, 8 hours, 10 hours, 12 hours, or 24 hrs. In some embodiments, the composition is capable of generating heat for at least 2 hours after application to the acupoint(s) for diabetes.

In some embodiments of the composition, optionally in combination with any or all of the above various embodiments, the heat generation component comprises a rare earth component. Such rare earth component can be a rare earth magnetic powder (e.g., samarium-cobalt magnetic material or neodymium based magnetic material) or a rare earth oxide, which can be any oxide of a rare earth element. Rare earth elements include lanthanide series elements and actinide series elements. Lanthanide series elements are Lanthanum, Cerium, Praseodymium, Neodymium, Promethium, Samarium, Europium, Gadolinium, Terbium, Dysprosium, Holmium, Erbium, Thulium, Ytterbium, and Lutetium; Actinide series elements are Actinium, Thorium, Protactinium, Uranium, Neptunium, Plutonium, Americium, Curium, Berkelium, Californium, Einsteinium, Fermium, Mendelevium, Nobelium, and Lawrencium.

In some embodiments of the composition, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of the composition, optionally in combination with any or all of the above various embodiments, the formulation is a patch comprising a cover. In some embodiments, the cover can be e.g., a plastic, rubber, or paper cover.

In another aspect of the present invention, it is provided a method of fabricating a composition for diabetes, comprising providing a heat generation component, and formulating the composition in a formulation for selective application to a set of acupoints or one or more acupoint(s) for diabetes (“acupoint(s) for diabetes”) in a patient. The composition generates heat upon application to the set of acupoints or one or more acupoint(s) for diabetes for a sufficient period of time to generate an effect to ameliorate or treat diabetes.

In some embodiments of the composition, the set of acupoints comprises the Shangwan, Zhongwan, Shenque, Zusanli, Weizhong, Ganshu, Danshu, and Shenshu acupoints of a diabetes patient.

In some embodiments of the method, optionally in combination with any or all of the above various embodiments, the composition is capable of generating heat for at least 10 minutes, e.g., 20 minutes, 30 minutes, 60 minutes (1 hour), 2 hours, 3 hours, 4 hours, 5 hours, 6 hours, 7 hours, 8 hours, 10 hours, 12 hours, 14 hours, or 24 hours. In some embodiments, the composition is capable of generating heat for at least 2 hours after application to the acupoint(s) for diabetes.

In some embodiments of the method, optionally in combination with any or all of the above various embodiments, the heat generation component comprises a rare earth component. Such rare earth component can be a rare earth magnetic powder (e.g., samarium-cobalt magnetic material or neodymium based magnetic material) or a rare earth oxide, which can be any oxide of a rare earth element. Rare earth elements include lanthanide series elements and actinide series elements. Lanthanide series elements are Lanthanum, Cerium, Praseodymium, Neodymium, Promethium, Samarium, Europium, Gadolinium, Terbium, Dysprosium, Holmium, Erbium, Thulium, Ytterbium, and Lutetium; Actinide series elements are Actinium, Thorium, Protactinium, Uranium, Neptunium, Plutonium, Americium, Curium, Berkelium, Californium, Einsteinium, Fermium, Mendelevium, Nobelium, and Lawrencium.

In some embodiments of the method, optionally in combination with any or all of the above various embodiments, the formulation is a patch comprising a cover. In some embodiments, the cover can be e.g., a plastic, rubber, or paper cover.

In a further embodiment of the present invention, it is provided a method of ameliorating or treating a diabetic condition, comprising applying a composition at least once per day to a set of acupoints or one or more acupoint(s) for diabetes (“acupoint(s) for diabetes”) in a diabetes patient, which composition comprising a heat generation component, wherein the composition is formulated in a formulation for selective application to the set of acupoints or one or more acupoint(s) for diabetes over the entire length of a prescribed treatment course, wherein the composition generates heat upon application to the set of acupoints or one or more acupoint(s) for diabetes for a sufficient period of time to generate an effect to ameliorate diabetes.

In some embodiments of the method, the composition is capable of generating heat for at least 10 minutes, e.g., 20 minutes, 30 minutes, 60 minutes (1 hour), 2 hours, 3 hours, 4 hours, 5 hours, 6 hours, 7 hours, 8 hours, 10 hours, 12 hours, 15 hours or a day. In some embodiments, the composition is capable of generating heat for at least 2 hours after application to the acupoint(s) for diabetes. In some embodiments, the composition is capable of generating heat for at least 2 hours after application to the acupoint(s) for diabetes.

In some embodiments of the method, optionally in combination with any or all of the above various embodiments, the heat generation component comprises a rare earth component. Such rare earth component can be a rare earth magnetic powder (e.g., samarium-cobalt magnetic material or neodymium based magnetic material) or a rare earth oxide, which can be any oxide of a rare earth element. Rare earth elements include lanthanide series elements and actinide series elements. Lanthanide series elements are Lanthanum, Cerium, Praseodymium, Neodymium, Promethium, Samarium, Europium, Gadolinium, Terbium, Dysprosium, Holmium, Erbium, Thulium, Ytterbium, and Lutetium; Actinide series elements are Actinium, Thorium, Protactinium, Uranium, Neptunium, Plutonium, Americium, Curium, Berkelium, Californium, Einsteinium, Fermium, Mendelevium, Nobelium, and Lawrencium.

In some embodiments of the method, optionally in combination with any or all of the above various embodiments, the heat generation component comprises a rare earth magnetic powder, saussurea in volucrata, musk, sea otter, chuanwu, caowu, lilac, menthol, meiyao, wujiapi, and maqianzhi.

In some embodiments of method, optionally in combination with any or all of the above various embodiments, the formulation is a patch comprising a cover. In some embodiments of method, the cover is a plastic, rubber, or paper cover.

In some embodiments of method, optionally in combination with any or all of the above various embodiments, the treatment course lasts for about 1 month, 3 months, 6 months, or 12 months.

As used herein, the term “heat generation component” can be any component capable generating heat upon exposure to moisture or air that provides a local temperature above body temperature (e.g., 37° C.) to about 50° C. Local temperature refers to the temperature of the skin area in contact with a composition disclosed herein. Such heat generation can last at least about 10 minutes, e.g., e.g., 20 minutes, 30 minutes, 60 minutes (1 hour), 2 hours, 3 hours, 4 hours, 5 hours, 6 hours, 7 hours, 8 hours 10 hours, 12 hours, 14 hours, or 24 hours. An example of the heat generating component is a chemical composition commonly in baby products or winter comfort products. For example, in some embodiments, the heat generation component can comprise iron powder, activated carbon, vermiculitm, and inorganic salt. In another embodiment, the heat generation component can comprise the heat generation component comprises a rare earth component. Such rare earth component can be a rare earth magnetic powder (e.g., samarium-cobalt magnetic material or neodymium based magnetic material) or a rare earth oxide, which can be any oxide of a rare earth element. Rare earth elements include lanthanide series elements and actinide series elements. Lanthanide series elements are Lanthanum, Cerium, Praseodymium, Neodymium, Promethium, Samarium, Europium, Gadolinium, Terbium, Dysprosium, Holmium, Erbium, Thulium, Ytterbium, and Lutetium; Actinide series elements are Actinium, Thorium, Protactinium, Uranium, Neptunium, Plutonium, Americium, Curium, Berkelium, Californium, Einsteinium, Fermium, Mendelevium, Nobelium, and Lawrencium.

As used herein, the term “a set of acupoints or one or more acupoint(s) for diabetes” shall mean any set of acupoints or one or more acupoint(s) identified as acupuncture target for ameliorating or treating a diabetic condition. In some embodiments, the set of acupoints or one or more acupoint(s) shall include the set of acupoints comprises the Shangwan, Zhongwan, Shenque, Zusanli, Weizhong, Ganshu, Danshu, and Shenshu acupoints of a diabetes patient (FIGS. 1A-1D).

As used herein, the term “diabetes” includes any of the above mentioned three types of diabetes.

### Acupuncture and Acupoints

Acupuncture, practiced for several thousand years in China (Veith I. The yellow emperor's classic of internal medicine. Williams & Wilkins: Baltimore; 1949. pp 58-76.), is increasingly used worldwide in the treatment of many disorders. An accumulating body of evidence summarized in a NIH Consensus Statement on Acupuncture (NIH Consensus Development Panel. Acupuncture. JAMA 1998; 280: 1518-1524) confirms that acupuncture treatment has beneficial effects for conditions ranging from postoperative dental pain to chemotherapy-associated emesis. It is also effective as an adjunctive modality for joint and muscle pain, addictions, and asthma, for example.

### Meridians and Acupoints

In Traditional Chinese Medicine (TCM), life force or ‘Qi’ (‘chee’) is thought to circulate within energy pathways or ‘meridians’ longitudinally throughout the body. There are 14 major meridians, corresponding (loosely) to the Western definition of ‘organs.’ Acupoints are specific locations on the body considered to be connected to these energy meridians (NIH Consensus Development Panel. Acupuncture. JAMA 1998; 280: 1518-1524; Vickers A, Zollman C. ABC of complementary medicine: acupuncture. BMJ 1999; 319: 973-976; and Mitchell ER. Fighting drug abuse with acupuncture: the treatment that works. Pacific View Press: Berkeley, Calif.; 1995). During illness Qi is thought to be out of balance, and stimulation of acupoints corrects this imbalance. Theoretically, an ‘excess’ or ‘deficiency’ of Qi can be ‘normalized’ by the specific manner of point stimulation.

In terms of traditional medicine, it is believed that acupuncture works to alter central nervous system neurotransmitter levels by stimulating peripheral nerves at acupoints. These stimulated nerves then carry the signals centrally (Stux G, Pomeranz B. Basics of acupuncture, 4th ed. Springer-Verlag: Berlin; 1998; Mann F. Acupuncture: cure of many diseases. Butterworth-Heinemann Ltd.: Oxford; 1992), including to the spinal cord, pituitary, and midbrain. Activated centers can then release neurochemicals: endorphins, monoamines, and cortisol (Stux G, Pomeranz B. Basics of acupuncture, 4th edn. Springer-Verlag: Berlin; 1998).

### Acupoint Stimulation

A large number of acupoints (365 points have been identified in Chinese acupuncture maps) show concentrations of peripheral nerve junctions, referred to as trigger points (Vickers A, Zollman C. ABC of complementary medicine: acupuncture. BMJ 1999; 319: 973-976). A variety of methods are used to stimulate acupoints (NIH Consensus Development Panel. Acupuncture. JAMA 1998: 280: 1518-1524; Stux G, Pomeranz B. Basics of acupuncture, 4th edn. Springer-Verlag: Berlin; 1998; Lao L. Acupuncture technique and devices. J Ahern Complem Med 1996; 2: 23-25). These include:
1. Needling or ‘traditional acupuncture’, in which fine stainless-steel needles are inserted through the skin to various depths, kept in place for varying lengths of time and/or further stimulated manually or electrically (‘electroacupuncture’).
2. Press needles or ‘staplepuncture’ in which short acupuncture needles are taped into place for extended periods with pressure being applied on a regular basis.
3. ‘Acupressure’ in which beads are massaged or pressed at specific locations.
4. ‘Moxibustion’ involving the application of heat, by burning small grain-sized pellets of combustible material on or near the acupoints.
5. ‘Cupping’ or using cups of various materials to create ‘negative pressure’ or a vacuum on the skin surface for increased blood circulation and point stimulation.
6. Transcutaneous electrical nerve stimulation (‘TENS’) which involves applying electrode pads to the skin surface which transmit a mild current into the acupoint.
   The teachings in the references of preceding paragraphs are incorporated herein by reference.

### Acupoints for Diabetes

For providing complementary treatment of diabetes using the device of invention, acupoints generally include Shangwan, Zhongwan, Shenque, Zusanli, Weizhong, Ganshu, Danshu, and Shenshu. These acupoints are fully described in the acupoints diagram generally available in the art. These acupoints are generally applicable to all types of diabetes.

For a particular type of diabetes, the acupoints include the above described set of acupoints and further, additional acupoints, which are generally available in the art.

For ameliorating or treating a diabetic condition of a particular patient, the acupoints include the above described set of acupoints and further, additional acupoints, which are generally known in the art.

### Formulations

The composition of invention can be formulated in any formulation suitable for topical, selective application to one or more acupoint(s) for diabetes. One example of the formulation is a paste. Another example is a patch. The patch can include having a cover or protective layer one side of which has a composition of invention placed thereonto. The cover can be plastic, paper, or a foil (e.g., aluminum containing foil). A thin film can be used to sandwich the composition with the cover so as to prevent the composition from exposure to moisture prior to application to acupoint(s) for diabetes of a user. A person of ordinary skill in the art can readily formulate a composition of invention into any of such formulations.

### EXAMPLES

### Example 1

### Complementary Treatment for Diabetes

A composition disclosed herein is formulated into a patch and applied to one or any of the acupoints for diabetes of a patient. The patch generates heat to cause the temperature of the one or more of the acupoints to have a temperature of about 50° C., which lasts several hours. The procedure is repeated over a course of 3 months. Improvement on patient conditions is observed.

While particular embodiments of the present invention have been shown and described, it will be obvious to those skilled in the art that changes and modifications can be made without departing from this invention in its broader aspects. Therefore, the claims are to encompass within their scope all such changes and modifications as fall within the true spirit and scope of this invention.

I claim:

1. A composition for diabetes, comprising a heat generation component, wherein the composition is formulated in a formulation for selective application to a set of acupoints or one or more acupoint(s) for diabetes (“acupoint(s) for diabetes”) in a patient, wherein the composition generates heat upon application to the acupoint(s) for diabetes for a sufficient period of time to generate an effect to ameliorate or treat diabetes.

2. The composition of claim 1, wherein the set of acupoints comprises the Shangwan, Zhongwan, Shenque, Zusanli, Weizhong, Ganshu, Danshu, and Shenshu acupoints of a diabetes patient.

3. The composition of claim 1 capable of generating heat for at least 1 hour after application to the set of acupoints or one or more acupoint(s) for diabetes.

4. The composition of claim 1 capable of generating heat for at least 2 hours after application to the set of acupoints or one or more acupoint(s) for diabetes.

5. The composition of claim 1, wherein the heat generation component comprises a rare earth component.

6. The composition of claim 5, wherein the heat generation component comprises a rare earth magnetic powder, saussurea in volucrata, musk, sea otter, chuanwu, caowu, lilac, menthol, meiyao, wujiapi, and maqianzhi.

7. The composition of claim 1 wherein the formulation is a patch comprising a cover.

8. The composition of claim 1 wherein the cover is a plastic, rubber, or paper cover.

9. A method of fabricating a composition for Diabetes, comprising providing a heat generation component, and formulating the composition in a formulation for selective application to a set of acupoints or one or more acupoint(s) for diabetes (“acupoint(s) for diabetes”) in a patient, wherein the composition generates heat upon application to the set of acupoints or one or more acupoint(s) for diabetes for a sufficient period of time to generate an effect to ameliorate or treat diabetes.

10. The method of claim 9, wherein the composition is capable of generating heat for at least 1 hour after application to the set of acupoints or one or more acupoint(s) for diabetes.

11. The method of claim 9, wherein the composition is capable of generating heat for at least 8 hours after application to the set of acupoints or one or more acupoint(s) for diabetes.

12. The method of claim 9, wherein the heat generation component comprises a rare earth component.

13. The method of claim 12, wherein the heat generation component comprises a rare earth magnetic powder, saussurea in volucrata, musk, sea otter, chuanwu, caowu, lilac, menthol, meiyao, wujiapi, and maqianzhi.

14. The method of claim 9, wherein the formulation is a patch comprising a cover.

15. A method of ameliorating or treating a diabetic condition, comprising:
applying a composition at least once per day to a set of acupoints or one or more acupoint(s) for diabetes (“acupoint(s) for diabetes”) in a diabetes patient, which composition comprising a heat generation component, wherein the composition is formulated in a formulation for selective application to the set of acupoints or one or more acupoint(s) for diabetes over the entire length of a prescribed treatment course, wherein the composition generates heat upon application to the set of acupoints or one or more acupoint(s) for diabetes for a sufficient period of time to generate an effect to ameliorate diabetes.

16. The method of claim 15, wherein the composition is capable of generating heat for at least 1 hour after application to the set of acupoints or one or more acupoint(s) for diabetes.

17. The method of claim 15, wherein the composition is capable of generating heat for at least 2 hours after application to the set of acupoints or one or more acupoint(s) for diabetes.

18. The method of claim 15, wherein the heat generation component comprises a rare earth component.

19. The method of claim 18, wherein the heat generation component comprises a rare earth magnetic powder, saussurea in volucrata, musk, sea otter, chuanwu, caowu, lilac, menthol, meiyao, wujiapi, and maqianzhi.

20. The method of claim 15, wherein the formulation is a patch comprising a cover.

21. The method of claim 15, wherein the cover is a plastic, rubber, or paper cover.

22. The method of claim 15, wherein the treatment course lasts fbr about 1 month, 3 months, 6 months, or 12 months.
